(19) United States
(12) Patent Application Publication
(10) Pub. No.: US 2022/0221433 A1
(43) Pub. Date: Jul. 14, 2022

(54) MASS SPECTROMETRY ASSAY METHODS FOR DETECTION OF METABOLITES

(71) Applicant: Metabolon, Inc., Morrisville, NC (US)

(72) Inventors: Richard J. Robinson, Morrisville, NC (US); Anne M. EVANS, Cary, NC (US); Elizaveta FREINKMAN, Cary, NC (US)

(21) Appl. No.: 17/611,639

(22) PCT Filed: May 8, 2020
(86) PCT No.: PCT/US20/31982

§ 371 Date: Nov. 16, 2021

Related U.S. Application Data
(60) Provisional application No. 62/964,683, filed on Jan. 23, 2020.
(60) Provisional application No. 62/855,004, filed on May 31, 2019.

(51) Int. Cl. G01N 30/72; G01N 30/06

(52) U.S. Cl. CPC G01N 30/7233; G01N 30/06; G01N 2030/027; G01N 2030/062; G01N 2560/00

(57) ABSTRACT

A method for determining in a sample, by liquid chromatography/mass spectrometry, the presence, absence, or amount of one or more or a plurality of analytes is described. The method comprises subjecting the sample to an ionization source under conditions suitable to produce one or more ions detectable by mass spectrometry from each of the one or more of the analytes; measuring, by liquid chromatography/mass spectrometry, the amount of the one or more ions from each of the one or more analytes; and using the measured amount of the one or more ions to determine the presence, absence, or amount of each of the one or more or plurality of analytes in the sample.

### CROSS-REFERENCE TO RELATED APPLICATIONS

This application claims the benefit of U.S. Provisional Patent Application No. 62/855,004, filed May 31, 2019, and U.S. Provisional Patent Application No. 62/964,683, filed Jan. 23, 2020, the entire contents of each of which are hereby incorporated herein by reference.

### BACKGROUND

The following information to describe the background of the invention is provided to assist the understanding of the invention and is not admitted to constitute or describe prior art to the invention.

Described herein are methods for determining the presence, absence, or amount of one or more or a plurality of analytes in a sample. The measured analytes may include one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, o-tyramine, phenylacetyl-beta-alanine, phenylacetyltaurine, phenylacetylvaline, 5-hydroxyindole glucuronide, chenodeoxycholic acid sulfate (1), deoxycholic acid (12 or 24)-sulfate, deoxycholic acid glucuronide, glycoursodeoxycholic acid sulfate (1), isoursodeoxycholate sulfate (1), ascorbic acid 3-sulfate, 1-(14 or 15-methyl)palmitoyl-GPC (a17:0 or i17:0), 3-hydroxyadipoylcarnitine, 3-hydroxymargaroylglycine, 4-methylnonanoylcarnitine, azelaoyltaurine, butyryltaurine, hexanoyltaurine, isobutyryltaurine, levulinoylcarnitine, undecenoylcarnitine (C11:1), 3,5-dichloro-2,6-dihydroxybenzoic acid, 3-bromo-5-chloro-2,6-dihydroxybenzoic acid, 2-methoxyhydroquinone glucuronide (2), 2-methoxyhydroquinone sulfate (2), 4-allylcatechol glucuronide, 4-allylcatechol sulfate, 4-ethylcatechol sulfate, 3-hydroxy-2-methylpyridine sulfate, 3-hydroxy-4-methylpyridine sulfate, 3-hydroxypyridine glucuronide, 5-hydroxy-2-methylpyridine sulfate, 2-iminopiperidine, thymidine sulfate (2), cyclo(ala-arg), cyclo(his-tyr), cyclo(his-val), N-acetylserine-valine-arginine, 4-vinylguaiacol glucuronide, maltol sulfate, methyl vanillate sulfate, butyrylputrescine, 5-androsten-3b,16a,17b-triol sulfate (1), 5-androsten-3b,16b,17a-triol sulfate (1), 5-androstenetriol disulfate, cortolone glucuronide, dehydroandrosterone glucuronide, (2-butoxyethoxy)acetic acid, dibutyl sulfosuccinate, 3-methylbutanol glucuronide, and combinations thereof.

### SUMMARY

In a first aspect of the invention, a method comprises determining the presence, absence, or amount of one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, o-tyramine, phenylacetyl-beta-alanine, phenylacetyltaurine, phenylacetylvaline, 5-hydroxyindole glucuronide, chenodeoxycholic acid sulfate (1), deoxycholic acid (12 or 24)-sulfate, deoxycholic acid glucuronide, glycoursodeoxycholic acid sulfate (1), isoursodeoxycholate sulfate (1), ascorbic acid 3-sulfate, 1-(14 or 15-methyl)palmitoyl-GPC (a17:0 or i17:0), 3-hydroxyadipoylcarnitine, 3-hydroxymargaroylglycine, 4-methylnonanoylcarnitine, azelaoyltaurine, butyryltaurine, hexanoyltaurine, isobutyryltaurine, levulinoylcarnitine, undecenoylcarnitine (C11:1), 3,5-dichloro-2,6-dihydroxybenzoic acid, 3-bromo-5-chloro-2,6-dihydroxybenzoic acid, 2-methoxyhydroquinone glucuronide (2), 2-methoxyhydroquinone sulfate (2), 4-allylcatechol glucuronide, 4-allylcatechol sulfate, 4-ethylcatechol sulfate, 3-hydroxy-2-methylpyridine sulfate, 3-hydroxy-4-methylpyridine sulfate, 3-hydroxypyridine glucuronide, 5-hydroxy-2-methylpyridine sulfate, 2-iminopiperidine, thymidine sulfate (2), cyclo(ala-arg), cyclo(his-tyr), cyclo(his-val), N-acetylserine-valine-arginine, 4-vinylguaiacol glucuronide, maltol sulfate, methyl vanillate sulfate, butyrylputrescine, 5-androsten-3b,16a,17b-triol sulfate (1), 5-androsten-3b,16b,17a-triol sulfate (1), 5-androstenetriol disulfate, cortolone glucuronide, dehydroandrosterone glucuronide, (2-butoxyethoxy)acetic acid, dibutyl sulfosuccinate, 3-methylbutanol glucuronide, and combinations thereof in a sample by liquid chromatography/mass spectrometry. In one embodiment, the method comprises subjecting the sample to an ionization source under conditions suitable to produce one or more ions detectable by mass spectrometry from each of the one or more or plurality of analytes. In another embodiment, the analytes are not derivatized prior to ionization. Methods to extract the analytes from biological samples and to chromatographically separate the analytes prior to detection by mass spectrometry are also provided.

In an embodiment, the one or more analytes may be categorized, for example, by biochemical association or chemical structure. In one example of this embodiment, the one or more analytes may be categorized based on biochemical association or chemical structure into a biochemical class. In another example, the one or more analytes may be further categorized into a subclass of the biochemical class.

In one embodiment, the analytes N2-acetyl, N6,N6-dimethyllysine, N-butyryl-leucine, N-butyryl-phenylalanine, N-succinyl-leucine, N-succinyl-phenylalanine, o-tyramine, phenylacetyl-beta-alanine, phenylacetyltaurine, phenylacetylvaline, and 5-hydroxyindole glucuronide may be categorized as amino acids. In another embodiment, the analyte N2-acetyl, N6,N6-dimethyllysine may be further categorized as an amino acid derivative; the analytes N-butyryl-leucine, N-butyryl-phenylalanine, N-succinyl-leucine, and N-succinyl-phenylalanine may be further categorized as short-chain fatty acid conjugates; the analytes phenylacetyl-beta-alanine, phenylacetyltaurine, and phenylacetylvaline may be further categorized as phenylacetic acid conjugates; o-tyramine may be further categorized as a chemical; and 5-hydroxyindole glucuronide may be further categorized as an aromatic glucuronide.

In another embodiment, the analytes chenodeoxycholic acid sulfate (1), deoxycholic acid (12 or 24)-sulfate, deoxycholic acid glucuronide, glycoursodeoxycholic acid sulfate (1), and isoursodeoxycholate sulfate (1) may be categorized as bile acids. In a further embodiment, the analytes chenodeoxycholic acid sulfate (1), deoxycholic acid (12 or 24)-sulfate, deoxycholic acid glucuronide, glycoursodeoxycholic acid sulfate (1), and isoursodeoxycholate sulfate (1) may be further categorized as sulfates or glucuronides of bile acids.

In another embodiment, the analyte ascorbic acid 3-sulfate may be categorized as being involved with cofactor metabolism. In a further embodiment, the analyte ascorbic acid 3-sulfate may be further categorized as a sulfated analyte of cofactor metabolism.

In another embodiment, the analyte 1-(14 or 15-methyl)palmitoyl-GPC (a17:0 or i17:0) may be categorized as a complex lipid.

In another embodiment, the analytes 3-hydroxyadipoylcarnitine, 3-hydroxymargaroylglycine, 4-methylnonanoylcarnitine, azelaoyltaurine, butyryltaurine, hexanoyltaurine, isobutyryltaurine, levulinoylcarnitine, and undecenoylcarnitine (C11:1) may be categorized as fatty acyl conjugates. In a further embodiment, the analytes 3-hydroxyadipoylcarnitine, 3-hydroxymargaroylglycine, 4-methylnonanoylcarnitine, azelaoyltaurine, butyryltaurine, hexanoyltaurine, isobutyryltaurine, levulinoylcarnitine, and undecenoylcarnitine (C11:1) may be further categorized as fatty acyl conjugates of carnitine, glycine, or taurine.

In another embodiment, the analytes 3,5-dichloro-2,6-dihydroxybenzoic acid, 3-bromo-5-chloro-2,6-dihydroxybenzoic acid, 2-methoxyhydroquinone glucuronide (2), 2-methoxyhydroquinone sulfate (2), 4-allylcatechol glucuronide, 4-allylcatechol sulfate, 4-ethylcatechol sulfate, 3-hydroxy-2-methylpyridine sulfate, 3-hydroxy-4-methylpyridine sulfate, 3-hydroxypyridine glucuronide, and 5-hydroxy-2-methylpyridine sulfate may be categorized as aromatic compounds. In a further embodiment, the analytes 3,5-dichloro-2,6-dihydroxybenzoic acid and 3-bromo-5-chloro-2,6-dihydroxybenzoic acid may be further categorized as halogenated benzoic acid derivatives; the analytes 2-methoxyhydroquinone glucuronide (2), 2-methoxyhydroquinone sulfate (2), 4-allylcatechol glucuronide, 4-allylcatechol sulfate, and 4-ethylcatechol sulfate may be further categorized as sulfates or glucuronides of phenols; and the analytes 3-hydroxy-4-methylpyridine sulfate, 3-hydroxypyridine glucuronide, and 5-hydroxy-2-methylpyridine sulfate may be further categorized as sulfates or glucuronides of pyridines.

In another embodiment, the analyte thymidine sulfate (2) may be categorized as a nucleotide. In a further embodiment, the analyte thymidine sulfate (2) may be further categorized as a sulfated nucleotide.

In another embodiment, the analytes N-acetylserine-valine-arginine, cyclo(ala-arg), cyclo(his-tyr), and cyclo(his-val) may be categorized as peptides. In a further embodiment, the analyte N-acetylserine-valine-arginine may be further categorized as a modified peptide; and the analytes cyclo(ala-arg), cyclo(his-tyr), and cyclo(his-val) may be further categorized as cyclic dipeptides.

In another embodiment, the analytes 4-vinylguaiacol glucuronide, maltol sulfate, and methyl vanillate sulfate may be categorized as plant metabolites. In a further embodiment, the analytes 4-vinylguaiacol glucuronide, maltol sulfate, and methyl vanillate sulfate may be further categorized as sulfates or glucuronides of plant metabolites.

In another embodiment, the analyte butyrylputrescine may be categorized as a polyamine. In a further embodiment, the analyte butyrylputrescine may be further categorized as a short chain fatty acid conjugate.

In another embodiment, the analytes 5-androsten-3b,16a,17b-triol sulfate (1), 5-androsten-3b,16b,17a-triol sulfate (1), 5-androstenetriol disulfate, cortolone glucuronide, and dehydroandrosterone glucuronide may be categorized as steroid hormone conjugates. In a further embodiment, the analytes 5-androsten-3b,16a,17b-triol sulfate (1), 5-androsten-3b,16b,17a-triol sulfate (1), 5-androstenetriol disulfate, cortolone glucuronide, and dehydroandrosterone glucuronide may be further categorized as sulfates or glucuronides of steroid hormones.

In another embodiment, the analytes 3-methylbutanol glucuronide, 2-iminopiperidine, (2-butoxyethoxy)acetic acid, and dibutyl sulfosuccinate may be categorized as xenobiotics. In a further embodiment, the analyte 3-methylbutanol glucuronide may be further categorized as a glucuronide of a xenobiotic; and the analytes 2-iminopiperidine, (2-butoxyethoxy)acetic acid, and dibutyl sulfosuccinate may be categorized as chemical xenobiotics.

In an embodiment, the mass spectrometry is tandem mass spectrometry.

In an embodiment, the method includes determining the presence, absence, or amount of one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 1-(14 or 15-methyl)palmitoyl-GPC (a17:0 or i17:0), 2-methoxyhydroquinone glucuronide (2), 2-methoxyhydroquinone sulfate (2), 3,5-dichloro-2,6-dihydroxybenzoic acid, 3-bromo-5-chloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-methylnonanoylcarnitine, 4-vinylguaiacol glucuronide, 5-hydroxy-2-methylpyridine sulfate, 5-hydroxyindole glucuronide, ascorbic acid 3-sulfate, azelaoyltaurine, butyrylputrescine, chenodeoxycholic acid sulfate (1), cortolone glucuronide, cyclo(ala-arg), cyclo(his-tyr), cyclo(his-val), dehydroandrosterone glucuronide, deoxycholic acid (12 or 24)-sulfate, deoxycholic acid glucuronide, dibutyl sulfosuccinate, glycoursodeoxycholic acid sulfate (1), hexanoyltaurine, isoursodeoxycholate sulfate (1), levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-methylbutanol glucuronide, 4-allylcatechol glucuronide, butyryltaurine, isobutyryltaurine, N-acetylserine-valine-arginine, and combinations thereof in a sample by liquid chromatography/mass spectrometry using a single injection.

In an embodiment, the method includes determining the presence, absence, or amount of one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 2-methoxyhydroquinone sulfate (2), 3,5-dichloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-vinylguaiacol glucuronide, 5-hydroxy-2-methylpyridine sulfate, 5-hydroxyindole glucuronide, ascorbic acid 3-sulfate, azelaoyltaurine, dehydroandrosterone glucuronide, deoxycholic acid glucuronide, dibutyl sulfosuccinate, hexanoyltaurine, levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-hydroxyadipoylcarnitine, 3-methylbutanol glucuronide, 4-allylcatechol glucuronide, butyryltaurine, isobutyryltaurine, N-acetylserine-valine-arginine, and combinations thereof in a sample by liquid chromatography/mass spectrometry using a single injection.

In an embodiment, the method includes determining the presence, absence, or amount of one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 2-iminopiperidine, 3-hydroxy-2-methylpyridine sulfate, 3-hydroxy-4-methylpyridine sulfate, 3-hydroxypyridine glucuronide, 4-ethylcatechol sulfate, 5-hydroxy-2-methylpyridine sulfate, ascorbic acid 3-sulfate, azelaoyltaurine, butyrylputrescine, dibutyl sulfosuccinate, hexanoyltaurine, levulinoylcarnitine, o-tyramine, phenylacetyl-beta-alanine, phenylacetyltaurine, phenylacetylvaline, 3-hydroxyadipoylcarnitine, butyryltaurine, isobutyryltaurine, N-acetylserine-valine-arginine, and combinations thereof in a sample by liquid chromatography/mass spectrometry using a single injection.

In an embodiment, the method includes determining the presence, absence, or amount of one or more or a plurality of analytes selected from the group consisting of N-butyryl-leucine, N-butyryl-phenylalanine, 1-(14 or 15-methyl)palmitoyl-GPC (a17:0 or i17:0), 3-hydroxymargaroylglycine, 4-methylnonanoylcarnitine, deoxycholic acid glucuronide, phenylacetylvaline, undecenoylcarnitine (C11:1), and combinations thereof in a sample by liquid chromatography/mass spectrometry using a single injection.

In some embodiments, the amounts of two or more, three or more, four or more, five or more, six or more, seven or more, ten or more, 20 or more, 30 or more, 40 or more, and up to 52 of the analytes are determined in a single injection. When the amounts of two or more analytes are determined, the two or more analytes may be referred to as a “plurality of analytes”.

In embodiments, the amount of the sample to be analyzed (i.e., sample volume or test sample volume) may be 10 μl to 200 μl. For example, the sample volume may be 10 μl, 15, 20, 25, 30, 40, 50 μl, 60, 70, 80, 90, 100, 120, 140, 160, 180 or 200 μl or any other volume between 10 and 200 μl.

### DETAILED DESCRIPTION

Methods are described for determining the presence, absence, or amount of one or more or a plurality of analytes selected from the group of metabolites consisting of N2-acetyl, N6,N6-dimethyllysine, N-butyryl-leucine, N-butyryl-phenylalanine, N-succinyl-leucine, N-succinyl-phenylalanine, o-tyramine, phenylacetyl-beta-alanine, phenylacetyltaurine, phenylacetylvaline, 5-hydroxyindole glucuronide, chenodeoxycholic acid sulfate (1), deoxycholic acid (12 or 24)-sulfate, deoxycholic acid glucuronide, glycoursodeoxycholic acid sulfate (1), isoursodeoxycholate sulfate (1), ascorbic acid 3-sulfate, 1-(14 or 15-methyl)palmitoyl-GPC (a17:0 or i17:0), 3-hydroxyadipoylcarnitine, 3-hydroxymargaroylglycine, 4-methylnonanoylcarnitine, azelaoyltaurine, butyryltaurine, hexanoyltaurine, isobutyryltaurine, levulinoylcarnitine, undecenoylcarnitine (C11:1), 3,5-dichloro-2,6-dihydroxybenzoic acid, 3-bromo-5-chloro-2,6-dihydroxybenzoic acid, 2-methoxyhydroquinone glucuronide (2), 2-methoxyhydroquinone sulfate (2), 4-allylcatechol glucuronide, 4-allylcatechol sulfate, 4-ethylcatechol sulfate, 3-hydroxy-2-methylpyridine sulfate, 3-hydroxy-4-methylpyridine sulfate, 3-hydroxypyridine glucuronide, 5-hydroxy-2-methylpyridine sulfate, 2-iminopiperidine, thymidine sulfate (2), cyclo(ala-arg), cyclo(his-tyr), cyclo(his-val), N-acetylserine-valine-arginine, 4-vinylguaiacol glucuronide, maltol sulfate, methyl vanillate sulfate, butyrylputrescine, 5-androsten-3b,16a,17b-triol sulfate (1), 5-androsten-3b,16b,17a-triol sulfate (1), 5-androstenetriol disulfate, cortolone glucuronide, dehydroandrosterone glucuronide, (2-butoxyethoxy)acetic acid, dibutyl sulfosuccinate, 3-methylbutanol glucuronide, and combinations thereof, in a sample. Mass spectrometric methods are described for determining the presence, absence, or amount of a one or more or a plurality of analytes in a sample. The methods may use a liquid chromatography step such as UPLC or HILIC to perform a separation (purification, enrichment) of selected analytes combined with methods of mass spectrometry, thereby providing a high-throughput assay system that is amenable to automation for quantifying one or more or a plurality of analytes in a sample.

Prior to describing this invention in further detail, the following terms are defined.

### Definitions

The term “solid phase extraction” refers to a sample preparation process where components of complex mixture (i.e., mobile phase) are separated according to their physical and chemical properties using solid particle chromatographic packing material (i.e. solid phase or stationary phase). The solid particle packing material may be contained in a cartridge type device (e.g. a column).

The term “separation” refers to the process of separating a complex mixture into its component molecules or metabolites. Common, exemplary laboratory separation techniques include electrophoresis and chromatography.

The term “chromatography” refers to a physical method of separation in which the components (i.e., chemical constituents) to be separated are distributed between two phases, one of which is stationary (stationary phase) while the other (the mobile phase) moves in a definite direction. The mobile phase may be gas (“gas chromatography”, “GC”) or liquid (“liquid chromatography”, “LC”). Chromatographic output data may be used in embodiments of the method described herein.

The term “liquid chromatography” or “LC” refers to a process of selective inhibition of one or more components of a fluid solution as the fluid uniformly moves through a column of a finely divided substance or through capillary passageways. The inhibition results from the distribution of the components of the mixture between one or more stationary phases and the mobile phase(s) as the mobile phase(s) move relative to the stationary phase(s). Examples of “liquid chromatography” include “Reverse phase liquid chromatography” or “RPLC”, “high performance liquid chromatography” or “HPLC”, “ultra-high performance liquid chromatography” or “UPLC” or “UHPLC”, or hydrophilic interaction chromatography or “HILIC”.

The term “retention time” refers to the elapsed time in a chromatography process since the introduction of the sample into the separation device. The retention time of a constituent of a sample refers to the elapsed time in a chromatography process between the time of injection of the sample into the separation device and the time that the constituent of the sample elutes (e.g., exits from) the portion of the separation device that contains the stationary phase.

The term “retention index” or “RI” of a sample component refers to a number, obtained by interpolation linear or logarithmic), relating the retention time or the retention factor of the sample component to the retention times of standards eluted before and after the peak of the sample component, a mechanism that uses the separation characteristics of known standards to remove systematic error.

The term “separation index” refers to a metric associated with chemical constituents separated by a separation technique. For chromatographic separation techniques, the separation index may be retention time or retention index. For non-chromatographic separation techniques, the separation index may be physical distance traveled by the chemical constituent.

As used herein, the terms “separation information” and “separation data” refer to data that indicates the presence or absence of chemical constituents with respect to the separation index. For example, separation data may indicate the presence of a chemical constituent having a particular mass eluting at a particular time. The separation data may indicate that the amount of the chemical constituent eluting over time rises, peaks, and then falls. A graph of the presence of the chemical constituent plotted over the separation index (e.g., time) may display a graphical peak. Thus, within the context of separation data, the terms “peak information” and “peak data” are synonymous with the terms “separation information” and “separation data”.

The term “Mass Spectrometry” (MS) refers to a technique for measuring and analyzing molecules that involves ionizing or ionizing and fragmenting a target molecule, then analyzing the ions, based on their mass/charge ratios, to produce a mass spectrum that serves as a “molecular fingerprint”. Determining the mass/charge ratio of an object may be done through means of determining the wavelengths at which electromagnetic energy is absorbed by that object. There are several commonly used methods to determine the mass to charge ratio of an ion, some measuring the interaction of the ion trajectory with electromagnetic waves, others measuring the time an ion takes to travel a given distance, or a combination of both. The data from these fragment mass measurements can be searched against databases to obtain identifications of target molecules.

The terms “operating in negative mode” or “operating in negative electrospray ionization (ESI) mode” or “operating in negative ionization mode” refer to those mass spectrometry methods where negative ions are generated and detected. The terms “operating in positive mode” or “operating in positive electrospray ionization (ESI) mode” or “operating in positive ionization mode” refer to those mass spectrometry methods where positive ions are generated and detected.

The term “mass analyzer” refers to a device in a mass spectrometer that separates a mixture of ions by their mass-to-charge (“m/z”) ratios.

The term “m/z” refers to the dimensionless quantity formed by dividing the mass number of an ion by its charge number. It has long been called the “mass-to-charge” ratio.

As used herein, the term “source” or “ionization source” refers to a device in a mass spectrometer that ionizes a sample to be analyzed. Examples of ionization sources include electrospray ionization (ESI), atmospheric pressure chemical ionization (APCI), heated electrospray ionization (HESI), atmospheric pressure photoionization (APPI), flame ionization detector (FID), matrix-assisted laser desorption ionization (MALDI), etc.

As used herein, the term “detector” refers to a device in a mass spectrometer that detects ions.

The term “ion” refers to any object containing a charge, which can be formed for example by adding electrons to or removing electrons from the object.

The term “mass spectrum” refers to a plot of data produced by a mass spectrometer, typically containing m/z values on x-axis and intensity values on y-axis.

The term “scan” refers to a mass spectrum that is associated with a particular separation index. For example, systems that use a chromatographic separation technique may generate multiple scans, each scan at a different retention time.

The term “run time”, refers to the time from sample injection to generation of the instrument data.

The term “tandem MS” refers to an operation in which a first MS step, called the “primary MS”, is performed, followed by performance of one or more of a subsequent MS step, generically referred to as “secondary MS”. In the primary MS, an ion, representing one (and possibly more than one) chemical constituent, is detected and recorded during the creation of the primary mass spectrum. The substance represented by the ion is subjected to a secondary MS, in which the substance of interest undergoes fragmentation in order to cause the substance to break into sub-components, which are detected and recorded as a secondary mass spectrum. In a true tandem MS, there is an unambiguous relationship between the ion of interest in the primary MS and the resulting peaks created during the secondary MS. The ion of interest in the primary MS corresponds to a “parent” or precursor ion, while the ions created during the secondary MS correspond to sub-components of the parent ion and are herein referred to as “daughter” or “product” ions.

Thus, tandem MS allows the creation of data structures that represent the parent-daughter relationship of chemical constituents in a complex mixture. This relationship may be represented by a tree-like structure illustrating the relationship of the parent and daughter ions to each other, where the daughter ions represent sub-components of the parent ion. Tandem MS may be repeated on daughter ions to determine “grand-daughter” ions, for example. Thus, tandem MS is not limited to two-levels of fragmentation, but is used generically to refer to multi-level MS, also referred to as “MS”. The term “MS/MS” is a synonym for “MS²”. For simplicity, the term “daughter ion” hereinafter refers to any ion created by a secondary or higher-order (i.e., not the primary) MS.

“Analyte”, “metabolite”, “biochemical” or “compound” refers to organic and inorganic small molecules. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000).

“Sample” can be any type of sample and may include a specimen or culture of natural or synthetic origin, including a complex mixture, an environmental sample, or a biological sample such as a plant sample or an animal sample. The complex mixture may be a synthetic formulation such as therapeutics and consumer goods, including cosmetics, supplements, food and drinks. The environmental sample refers to environmental material such as surface matter, soil, water, and industrial samples, as well as samples obtained from food and dairy processing instruments, apparatus, equipment, utensils, disposable and non-disposable items. The animal sample may be from a mammal such as, for example, a human, a mouse, a non-human primate, a rabbit or other mammal, or a non-mammal sample such as, for example, a drosophila or zebrafish sample. The biological sample of interest can include blood, plasma, serum, feces, isolated lipoprotein fraction, saliva, urine, lymph fluid, and cerebrospinal fluid, a tissue sample, a cellular sample, a skin sample, a plant sample, or a fungus sample. The biological sample may contain any biological material suitable for detecting the desired analytes and may comprise cellular and/or non-cellular material. The biological sample can also include cell cultures and culture and fermentation media, liquid and solid food and feed products and ingredients such as dairy items, grains, vegetables, meat and meat by-products, and waste. The sample can be isolated from any suitable biological tissue or fluid such as, for example, blood, blood plasma, serum, skin, epidermal tissue, adipose tissue, aortic tissue, liver tissue, urine, cerebral spinal fluid, crevicular fluid, amniotic fluid, or cell samples. The sample can be, for example, a dried blood spot where blood samples are blotted and dried on filter paper. In another the example, the sample can be isolated from a skin tape such as Sebutape®. The sample may be a control (reference) sample having known amounts of one or more analytes or a test (experimental) sample wherein the presence, absence or amount of one or more analytes is not known or needs to be determined.

“Subject” means any animal, but is preferably a mammal, such as, for example, a human, monkey, non-human primate, mouse, dog, rabbit or rat.

“Internal Standard” is a known concentration of an analyte that is added to every sample analyzed. As used herein, internal standard refers to “recovery standard” and “reconstitution standard”.

The term “recovery standard” refers to an internal standard that is added to a sample at a known concentration during analyte extraction and is used to assess the quality of sample extraction.

The term “reconstitution standard” refers to an internal standard that is added to a sample at a known concentration after sample extraction and is used to monitor instrument performance.

### I. Sample Preparation and Quality Control (QC)

Sample extracts containing analytes are prepared by isolating the analytes away from the macromolecules (e.g., proteins, nucleic acids, lipids) that may be present in the sample. Some or all analytes in a sample may be bound to proteins. Various methods may be used to disrupt the interaction between analyte(s) and protein prior to MS analysis. For example, the analytes may be extracted from a sample to produce a liquid extract, while the proteins that may be present are precipitated and removed. Proteins may be precipitated using, for example, a solution of ethyl acetate or methanol. To precipitate the proteins in the sample, an ethyl acetate or methanol solution is added to the sample, then the mixture may be spun in a centrifuge to separate the liquid supernatant, which contains the extracted analytes, from the precipitated proteins. In one example, a solution of methanol and water may be used to extract analytes from the sample.

In other embodiments, analytes may be released from protein without precipitating the protein. For example, a formic acid solution may be added to the sample to disrupt the interaction between protein and analyte. Alternatively, ammonium sulfate, a solution of formic acid in ethanol, or a solution of formic acid in methanol may be added to the sample to disrupt ionic interactions between protein and analyte without precipitating the protein.

In some embodiments the extract may be subjected to various methods including liquid chromatography, electrophoresis, filtration, centrifugation, and affinity separation as described herein to purify or enrich the amount of the selected analyte relative to one or more other components in the sample.

### II. Chromatography

Prior to mass spectrometry, the analyte extract may be subjected to one or more separation methods such as electrophoresis, filtration, centrifugation, affinity separation, or chromatography. In one embodiment the separation method may comprise liquid chromatography (LC), including, for example, ultra high performance LC (UHPLC, UPLC).

In some embodiments, UHPLC may be conducted using a reversed phase column chromatographic system, hydrophilic interaction chromatography (HILIC), or a mixed phase column chromatographic system.

The column heater (or column manager) for LC may be set at a temperature of from about 25° C. to about 80° C. For example, the column heater may be set at about 30° C., 40° C., 50° C., 60° C., 70° C., etc.

In an example, UHPLC may be conducted using a HILIC system. In another example, UHPLC may be conducted using a reversed phase column chromatographic system. The system may comprise two or more mobile phases. Mobile phases may be referred to as, for example, mobile phase A, mobile phase B, mobile phase A′, and mobile phase B′.

In an exemplary embodiment using two mobile phases, A and B, mobile phase A may comprise ammonium bicarbonate in water, and mobile phase B may comprise ammonium bicarbonate in methanol and water. The concentration of ammonium bicarbonate may range from 1 mM to 10 mM, and the concentration of methanol may range from 1% to 99%. In some examples, the concentration of ammonium bicarbonate in mobile phase A may be 3.0, 3.5, 4.0, 4.5, 5.0, 5.5, 6.0, 6.5, 7.0, 7.5, 8.0, or 8.5 mM. In some examples, the concentration of ammonium bicarbonate in mobile phase B may be 3.0, 3.5, 4.0, 4.5, 5.0, 5.5, 6.0, 6.5, 7.0, 7.5, 8.0, or 8.5 mM, and the concentration of methanol may be 90, 91, 92, 93, 94, 95, 96, 97, 98, or 99%.

In one example, linear gradient elution may be used for chromatography. The starting conditions for linear gradient elution may include the concentration of a mobile phase (e.g., mobile phase B) and/or the flow rate of a mobile phase through the column (e.g., mobile phase B). The starting conditions may be optimized for the separation and/or retention of one or more analytes. The gradient conditions may also be optimized for the separation and/or retention of analytes and may vary depending on the flow rate selected. For example, initial conditions may be 0.5% mobile phase B and 350 μL/min flow rate. Mobile phase B may be increased to 50-75%, increased to about 75-99% at about 4.5 minutes, maintained for 1-2 min. Mobile phase B may revert to 0.5% at 5.7 minutes where it may be maintained for less than a minute before the next sample injection. The total run time may be 6.5 minutes or less.

In another embodiment, mobile phase A may comprise ammonium formate, acetonitrile, methanol, and water, and mobile phase B may comprise ammonium formate and acetonitrile. The concentration of ammonium formate may range from 0.1 mM to 100 mM, and the concentration of acetonitrile may range from 0% to 100%. In some embodiments, the pH of the mobile phase may be basic and range from pH 8 to pH 14. In some examples, the concentration of ammonium formate in mobile phase A may be 1 mM, 5 mM, 10 mM, 15 mM, 20 mM, 25 mM, or 50 mM, and the concentration of acetonitrile may be 60, 70, 80, or 90%. In other examples, the concentration of ammonium formate in mobile phase B may be 1 mM, 5 mM, 10 mM, 15 mM, 20 mM, 25 mM, or 50 mM, and the concentration of acetonitrile may be 30%, 40%, 50%, or 60%. Linear gradient elution may be used for chromatography. For example, initial conditions may be 5% mobile phase B and 500 μL/min flow rate. Mobile phase B may be increased to about 40-60% at about 3-4 minutes, increased to about 75-99% at 4-6 minutes, and maintained for about 1 min. Mobile phase B may revert to 5% at 6-7 min where it may be maintained for about one minute before the next sample injection. The total run time may be 6.8 minutes or less.

In yet another example, mobile phase A may comprise perfluoropentanoic acid (PFPA), formic acid, and water, and mobile phase B may comprise PFPA, formic acid, and methanol. The concentration of PFPA may be from about 0.01 to about 0.50%, and the concentration of formic acid may be from about 0.001 to about 1.0%. In some examples, the concentration of PFPA in mobile phase A may be 0.03, 0.04, 0.05, 0.06, 0.07, 0.08, 0.09, 0.1, 0.2, or 0.3%, and the concentration of formic acid may be 0.001, 0.005, 0.1, 0.2, 0.3, 0.4, or 0.5%. In other examples, the concentration of PFPA in mobile phase B may be 0.03, 0.04, 0.05, 0.06, 0.07, 0.08, 0.09, 0.1, 0.2, or 0.3%, and the concentration of formic acid may be 0.001, 0.005, 0.1, 0.2, 0.3, 0.4, or 0.5%. Linear gradient elution may be used for chromatography. For example, initial conditions may be 5% mobile phase B and 350 Lt/min flow rate. Mobile phase B may be increased to about 75-99% at about 3.3 minutes. Mobile phase B may revert to 5% by 3.4 min where it may be maintained for less than one minute before the next sample injection. The total run time may be 3.4 minutes or less.

In yet another example, mobile phase A may comprise perfluoropentanoic acid (PFPA), formic acid, and water, and mobile phase B may comprise PFPA, formic acid, acetonitrile, and methanol. The concentration of PFPA may be from about 0.01 to about 0.50%, the concentration of formic acid may be from about 0.001 to about 1.0%, the concentration of methanol may be from 1 to 99%, and the concentration of acetonitrile may be from 1-99%. In some examples, the concentration of PFPA in mobile phase A may be 0.03, 0.04, 0.05, 0.06, 0.07, 0.08, 0.09, 0.1, 0.2, or 0.3%, and the concentration of formic acid may be 0.001, 0.005, 0.1, 0.2, 0.3, 0.4, or 0.5%. In other examples, the concentration of PFPA in mobile phase B may be 0.03, 0.04, 0.05, 0.06, 0.07, 0.08, 0.09, 0.1, 0.2, or 0.3%, the concentration of formic acid may be 0.001, 0.005, 0.1, 0.2, 0.3, 0.4, or 0.5%, the concentration of methanol may be 20%, 30%, 40%, 50%, 60%, or 70%, and the concentration of acetonitrile may be 20%, 30%, 40%, 50%, 60%, or 70%. Linear gradient elution may be used for chromatography. For example, initial conditions may be 40% mobile phase B and 600 μL/min flow rate. Mobile phase B may be increased to about 99% at one minute and may be maintained for less than 2.5 minutes. Mobile phase B may revert to 40% by about 3.4 min. The total run time may be 3.4 minutes or less.

### III. Mass Spectrometry

One or more analytes may be ionized by, for example, mass spectrometry. Mass spectrometry is performed using a mass spectrometer that includes an ionization source for ionizing the fractionated sample and creating charged molecules for further analysis. Ionization of the sample may be performed by, for example, electrospray ionization (ESI). Other ion sources may include, for example, atmospheric pressure chemical ionization (APCI), heated electrospray ionization (HESI), atmospheric pressure photoionization (APPI), flame ionization detector (FID), or matrix-assisted laser desorption ionization (MALDI). The choice of ionization method may be determined based on a number of considerations. Exemplary considerations include the analyte to be measured, type of sample, type of detector, and the choice of positive or negative mode.

The one or more analytes may be ionized in positive or negative mode to create one or more ions. For example, the analytes N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 1-(14 or 15-methyl)palmitoyl-GPC (a17:0 or i17:0), 2-methoxyhydroquinone glucuronide (2), 2-methoxyhydroquinone sulfate (2), 3,5-dichloro-2,6-dihydroxybenzoic acid, 3-bromo-5-chloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-methylnonanoylcarnitine, 4-vinylguaiacol glucuronide, 5-hydroxy-2-methylpyridine sulfate, 5-hydroxyindole glucuronide, ascorbic acid 3-sulfate, azelaoyltaurine, butyrylputrescine, chenodeoxycholic acid sulfate (1), cortolone glucuronide, cyclo(ala-arg), cyclo(his-tyr), cyclo(his-val), dehydroandrosterone glucuronide, deoxycholic acid (12 or 24)-sulfate, deoxycholic acid glucuronide, dibutyl sulfosuccinate, glycoursodeoxycholic acid sulfate (1), hexanoyltaurine, isoursodeoxycholate sulfate (1), levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-methylbutanol glucuronide, 4-allylcatechol glucuronide, butyryltaurine, isobutyryltaurine, N-acetylserine-valine-arginine, 3-hydroxyadipoylcarnitine, and combinations thereof may be ionized in negative mode.

In yet another example, the analytes N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 1-(14 or 15-methyl)palmitoyl-GPC (a17:0 or i17:0), 2-methoxyhydroquinone glucuronide (2), 2-methoxyhydroquinone sulfate (2), 3,5-dichloro-2,6-dihydroxybenzoic acid, 3-bromo-5-chloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-methylnonanoylcarnitine, 4-vinylguaiacol glucuronide, 5-hydroxy-2-methylpyridine sulfate, 5-hydroxyindole glucuronide, ascorbic acid 3-sulfate, azelaoyltaurine, butyrylputrescine, chenodeoxycholic acid sulfate (1), cortolone glucuronide, cyclo(ala-arg), cyclo(his-tyr), cyclo(his-val), dehydroandrosterone glucuronide, deoxycholic acid (12 or 24)-sulfate, deoxycholic acid glucuronide, dibutyl sulfosuccinate, glycoursodeoxycholic acid sulfate (1), hexanoyltaurine, isoursodeoxycholate sulfate (1), levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-methylbutanol glucuronide, 4-allylcatechol glucuronide, butyryltaurine, isobutyryltaurine, N-acetylserine-valine-arginine, 3-hydroxyadipoylcarnitine, and combinations thereof may be ionized in positive mode.

In one example, one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 1-(14 or 15-methyl)palmitoyl-GPC (a17:0 or i17:0), 2-methoxyhydroquinone glucuronide (2), 2-methoxyhydroquinone sulfate (2), 3,5-dichloro-2,6-dihydroxybenzoic acid, 3-bromo-5-chloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-methylnonanoylcarnitine, 4-vinylguaiacol glucuronide, 5-hydroxy-2-methylpyridine sulfate, 5-hydroxyindole glucuronide, ascorbic acid 3-sulfate, azelaoyltaurine, butyrylputrescine, chenodeoxycholic acid sulfate (1), cortolone glucuronide, cyclo(ala-arg), cyclo(his-tyr), cyclo(his-val), dehydroandrosterone glucuronide, deoxycholic acid (12 or 24)-sulfate, deoxycholic acid glucuronide, dibutyl sulfosuccinate, glycoursodeoxycholic acid sulfate (1), hexanoyltaurine, isoursodeoxycholate sulfate (1), levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-methylbutanol glucuronide, 4-allylcatechol glucuronide, butyryltaurine, isobutyryltaurine, N-acetylserine-valine-arginine, and combinations thereof may be ionized in negative mode and may be measured in a single injection.

In another example, one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 2-methoxyhydroquinone sulfate (2), 3,5-dichloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-vinylguaiacol glucuronide, 5-hydroxy-2-methylpyridine sulfate, 5-hydroxyindole glucuronide, ascorbic acid 3-sulfate, azelaoyltaurine, dehydroandrosterone glucuronide, deoxycholic acid glucuronide, dibutyl sulfosuccinate, hexanoyltaurine, levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-hydroxyadipoylcarnitine, 3-methylbutanol glucuronide, 4-allylcatechol glucuronide, butyryltaurine, isobutyryltaurine, N-acetylserine-valine-arginine, and combinations thereof may be ionized in negative mode and may be measured in a single injection.

In another example, one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 2-iminopiperidine, 3-hydroxy-2-methylpyridine sulfate, 3-hydroxy-4-methylpyridine sulfate, 3-hydroxypyridine glucuronide, 4-ethylcatechol sulfate, 5-hydroxy-2-methylpyridine sulfate, ascorbic acid 3-sulfate, azelaoyltaurine, butyrylputrescine, dibutyl sulfosuccinate, hexanoyltaurine, levulinoylcarnitine, o-tyramine, phenylacetyl-beta-alanine, phenylacetyltaurine, phenylacetylvaline, 3-hydroxyadipoylcarnitine, butyryltaurine, isobutyryltaurine, N-acetylserine-valine-arginine, and combinations thereof may be ionized in positive mode and may be measured in a single injection.

In another example, one or more or a plurality of analytes selected from the group consisting of N-butyryl-leucine, N-butyryl-phenylalanine, 1-(14 or 15-methyl)palmitoyl-GPC (a17:0 or i17:0), 3-hydroxymargaroylglycine, 4-methylnonanoylcarnitine, deoxycholic acid glucuronide, phenylacetylvaline, undecenoylcarnitine (C11:1), and combinations thereof may be ionized in positive mode and may be measured in a single injection.

Mass spectrometer instrument settings may be optimized for the given analysis method and/or for the particular mass spectrometer used. The instrument may use various gases, for example, nitrogen, helium, argon, or zero air. In an embodiment, mass spectrometry may be performed using Thermo Q-Exactive mass spectrometers. In one example, the mass spectrometer may be operated in negative electrospray ionization (ESI) mode. The ionspray voltage setting may range from about −0.5 kV to about −5.5 kV; in one embodiment the voltage may be set at −3.2 kV. The source temperature may range from about 200° C. to about 500° C.; in one embodiment the source temperature may be set at 300° C. The capillary temperature may range from about 200° C. to about 500° C.; in one embodiment the capillary temperature may be set at 300° C. The sheath gas may range from about 40 to about 90 units; in one embodiment the sheath gas is set at 70 units. The auxiliary gas may range from about 0 to about 90 units. In one embodiment the auxiliary gas may be set at 25. The S-lens radio frequency (RF) level may range from 20 to 60; in one embodiment, the S-lens RF level may be set at 40. The stepped collision energy may range from about −30 V to about −90 V.

In another example, the MS instrument may be operated in negative ESI mode. Ionspray voltage settings may range from −0.5 kV to −5.5 kV; in one embodiment the voltage may be set at −3.2 kV. The source temperature may range from about 200° C. to about 500° C.; in one embodiment the source temperature may be set at 300° C. The capillary temperature may range from about 200° C. to about 500° C.; in one embodiment the capillary temperature may be set at 300° C. The sheath gas may range from about 40 to about 90 units; in one embodiment the sheath gas is set at 70 units. The auxiliary gas may range from about 0 to about 90 units. In one embodiment the auxiliary gas may be set at 20. The S-lens radio frequency (RF) level may range from 20 to 60; in one embodiment, the S-lens RF level may be set at 40. The stepped collision energy (CE) may range from about −30 V to about −90 V.

In another example, the MS instrument may be operated in positive ESI mode. Ionspray voltage settings may range from 0.5 kV to 6.0V; in one embodiment the voltage may be set at 4.0 kV. The source temperature may range from about 200° C. to about 500° C.; in one embodiment the source temperature may be set at 300° C. The capillary temperature may range from about 100° C. to about 500° C.; in one embodiment the capillary temperature may be set at 250° C. The sheath gas may range from about 40 to about 90 units; in one embodiment the sheath gas is set at 70 units. The auxiliary gas may range from about 0 to about 90 units. In one embodiment the auxiliary gas may be set at 15. The S-lens radio frequency (RF) level may range from 20 to 60; in one embodiment, the S-lens RF level may be set at 40. The stepped collision energy may range from about 30 V to about 90 V.

In another example, the MS instrument may be operated in positive ESI mode. Ionspray voltage settings may range from 0.5 kV to 6.0V; in one embodiment the voltage may be set at 4.2 kV. The source temperature may range from about 200° C. to about 500° C.; in one embodiment the source temperature may be set at 400° C. The capillary temperature may range from about 200° C. to about 500° C.; in one embodiment the capillary temperature may be set at 350° C. The sheath gas may range from about 20 to about 90 units; in one embodiment the sheath gas is set at 45 units. The auxiliary gas may range from about 0 to about 90 units. In one embodiment the auxiliary gas may be set at 30. The S-lens radio frequency (RF) level may range from 20 to 60; in one embodiment, the S-lens RF level may be set at 40. The stepped collision energy may range from about 30 V to about 90 V.

After a sample has been ionized, the positively or negatively charged ions may be analyzed to determine a mass-to-charge ratio. Exemplary suitable analyzers for determining mass-to-charge ratios include quadrupole analyzers, ion trap analyzers, and time of flight analyzers. The ions may be detected using, a full scanning mode, for example, electrospray ionization (ESI).

Analysis results may include data produced by tandem MS. In exemplary embodiments, tandem MS may be accurate-mass tandem MS. For example, the accurate-mass tandem mass spectrometry may use an orbitrap analyzer. Tandem MS allows the creation of data structures that represent the parent-daughter relationship of chemical constituents in a complex mixture. This relationship may be represented by a tree-like structure illustrating the relationship of the parent and daughter ions to each other, where the daughter ions represent sub-components of the parent ion.

For example, a primary mass spectrum may contain five distinct ions, which may be represented as five graphical peaks. Each ion in the primary MS may be a parent ion. Each parent ion may be subjected to a secondary MS that produces a mass spectrum showing the daughter ions for that particular parent ion.

The parent/daughter relationship may be extended to describe the relationship between separated components (e.g., components eluting from the chromatography state) and ions detected in the primary MS, and to the relationship between the sample to be analyzed and the separated components.

The mass spectrometer typically provides the user with an ion scan (i.e., a relative abundance of each ion with a particular mass/charge (m/z) over a given range). Mass spectrometry data may be processed using software that allows for peak detection and integration. The output from this processing may generate a list of m/z ratios, retention times and area under the curve values. The software may also specify a criterion for peak detection such as, for example, thresholds for signal to noise ratio, height and width.

### IV. Kit

A kit for assaying one or more or a plurality of the analytes selected from the group consisting of N2-acetyl, N6,N6-dimethyllysine, N-butyryl-leucine, N-butyryl-phenylalanine, N-succinyl-leucine, N-succinyl-phenylalanine, o-tyramine, phenylacetyl-beta-alanine, phenylacetyltaurine, phenylacetylvaline, 5-hydroxyindole glucuronide, chenodeoxycholic acid sulfate (1), deoxycholic acid (12 or 24)-sulfate, deoxycholic acid glucuronide, glycoursodeoxycholic acid sulfate (1), isoursodeoxycholate sulfate (1), ascorbic acid 3-sulfate, 1-(14 or 15-methyl)palmitoyl-GPC (a17:0 or i17:0), 3-hydroxyadipoylcarnitine, 3-hydroxymargaroylglycine, 4-methylnonanoylcarnitine, azelaoyltaurine, butyryltaurine, hexanoyltaurine, isobutyryltaurine, levulinoylcarnitine, undecenoylcarnitine (C11:1), 3,5-dichloro-2,6-dihydroxybenzoic acid, 3-bromo-5-chloro-2,6-dihydroxybenzoic acid, 2-methoxyhydroquinone glucuronide (2), 2-methoxyhydroquinone sulfate (2), 4-allylcatechol glucuronide, 4-allylcatechol sulfate, 4-ethylcatechol sulfate, 3-hydroxy-2-methylpyridine sulfate, 3-hydroxy-4-methylpyridine sulfate, 3-hydroxypyridine glucuronide, 5-hydroxy-2-methylpyridine sulfate, 2-iminopiperidine, thymidine sulfate (2), cyclo(ala-arg), cyclo(his-tyr), cyclo(his-val), N-acetylserine-valine-arginine, 4-vinylguaiacol glucuronide, maltol sulfate, methyl vanillate sulfate, butyrylputrescine, 5-androsten-3b,16a,17b-triol sulfate (1), 5-androsten-3b,16b,17a-triol sulfate (1), 5-androstenetriol disulfate, cortolone glucuronide, dehydroandrosterone glucuronide, (2-butoxyethoxy)acetic acid, dibutyl sulfosuccinate, 3-methylbutanol glucuronide, and combinations thereof, is described herein. For example, a kit may include known concentrations of one or more internal standards to use for recovery standards or reconstitution standards in amounts sufficient for one or more assays, chromatography column(s), packaging material, and instructions for use. In exemplary embodiments, the internal standards may be labeled (such as isotopically labeled or radiolabeled), the kit may comprise pre-made mobile phase solutions, and/or the kit may comprise mobile phase reagents and instructions to prepare the mobile phase solutions. Kits may also comprise instructions recorded in tangible form (e.g. on paper such as, for example, an instruction booklet or an electronic medium) for using the reagents to measure the one or more analytes.

In one embodiment, a kit for assaying one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 1-(14 or 15-methyl)palmitoyl-GPC (a17:0 or i17:0), 2-methoxyhydroquinone glucuronide (2), 2-methoxyhydroquinone sulfate (2), 3,5-dichloro-2,6-dihydroxybenzoic acid, 3-bromo-5-chloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-methylnonanoylcarnitine, 4-vinylguaiacol glucuronide, 5-hydroxy-2-methylpyridine sulfate, 5-hydroxyindole glucuronide, ascorbic acid 3-sulfate, azelaoyltaurine, butyrylputrescine, chenodeoxycholic acid sulfate (1), cortolone glucuronide, cyclo(ala-arg), cyclo(his-tyr), cyclo(his-val), dehydroandrosterone glucuronide, deoxycholic acid (12 or 24)-sulfate, deoxycholic acid glucuronide, dibutyl sulfosuccinate, glycoursodeoxycholic acid sulfate (1), hexanoyltaurine, isoursodeoxycholate sulfate (1), levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-methylbutanol glucuronide, 4-allylcatechol glucuronide, butyryltaurine, isobutyryltaurine, N-acetylserine-valine-arginine, and combinations thereof is provided.

In another embodiment, a kit for assaying one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 1-(14 or 15-methyl)palmitoyl-GPC (a17:0 or i17:0), 2-methoxyhydroquinone glucuronide (2), 2-methoxyhydroquinone sulfate (2), 3,5-dichloro-2,6-dihydroxybenzoic acid, 3-bromo-5-chloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-methylnonanoylcarnitine, 4-vinylguaiacol glucuronide, 5-hydroxy-2-methylpyridine sulfate, 5-hydroxyindole glucuronide, ascorbic acid 3-sulfate, azelaoyltaurine, butyrylputrescine, chenodeoxycholic acid sulfate (1), cortolone glucuronide, cyclo(ala-arg), cyclo(his-tyr), cyclo(his-val), dehydroandrosterone glucuronide, deoxycholic acid (12 or 24)-sulfate, deoxycholic acid glucuronide, dibutyl sulfosuccinate, glycoursodeoxycholic acid sulfate (1), hexanoyltaurine, isoursodeoxycholate sulfate (1), levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-methylbutanol glucuronide, 4-allylcatechol glucuronide, butyryltaurine, isobutyryltaurine, N-acetylserine-valine-arginine, and combinations thereof is provided.

In another embodiment, a kit for assaying one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 2-iminopiperidine, 3-hydroxy-2-methylpyridine sulfate, 3-hydroxy-4-methylpyridine sulfate, 3-hydroxypyridine glucuronide, 4-ethylcatechol sulfate, 5-hydroxy-2-methylpyridine sulfate, ascorbic acid 3-sulfate, azelaoyltaurine, butyrylputrescine, dibutyl sulfosuccinate, hexanoyltaurine, levulinoylcarnitine, o-tyramine, phenylacetyl-beta-alanine, phenylacetyltaurine, phenylacetylvaline, 3-hydroxyadipoylcarnitine, butyryltaurine, isobutyryltaurine, N-acetylserine-valine-arginine, and combinations thereof is provided.

In another embodiment, a kit for assaying one or more or a plurality of analytes selected from the group consisting of N-butyryl-leucine, N-butyryl-phenylalanine, 1-(14 or 15-methyl)palmitoyl-GPC (a17:0 or i17:0), 3-hydroxymargaroylglycine, 4-methylnonanoylcarnitine, deoxycholic acid glucuronide, phenylacetylvaline, undecenoylcarnitine (C11:1), and combinations thereof is provided.

### EXAMPLES

### I. Sample Preparation

Sample preparation was carried out in a 96-well plate. 100 ul of sample was plated in the appropriate well of the 96-well plate. To extract the analytes from the samples, 500 μL of 100% methanol, containing a mixture of recovery standards used to determine the quality of the extraction procedure, was added to the samples. The samples were then mixed via agitation on a Genogrinder at 675SPM for 2 minutes. The plates were then spun in a centrifuge at 2800 rpm for 10 min (1100 G) in order to pellet the precipitated protein. An aliquot of 85 μl supernatant was transferred to each of 5 new plates (4×384 well 120 μL square well plates and 1×96 well PCR plate). The aliquots of methanolic extract were dried under nitrogen until dry.

For sample analysis, the plated, dried sample extract was reconstituted in reconstitution solvent containing reconstitution standards. The reconstitution solvent and reconstitution standards were optimized for the given analytical method. Reconstitution solvents were as follows: For Method 1 (LC/MS Negative), 6.5 mM Ammonium Bicarbonate; For Method 2 (LC Polar/MS Negative), 15% H₂O/5% MeOH/80% ACN (10 mM Ammonium Formate) pH 10.8; For Method 3 (LC/MS Positive Early), 0.1% Formic acid, 0.5% PFPA in water; For Method 4 (LC/MS Positive Late), 90% Isopropanol/10% H₂O 0.1% Formic acid 0.05% PFPA. The reconstitution standards were used to align chromatographic peaks and to monitor instrument performance. The sample plates were sealed and each plate was vortexed at 2200 rpm for 1 min and sonicated in a room temperature water bath for 5 minutes (30 minutes for Method 2 plates). Following the sonication, the plates were spun for 5 min at 2800 rpm (1100 G) to pellet any particulates in the sample.

### II. Data Processing and Analysis

For each set of samples for each method, relative standard deviation (RSD) of peak area was calculated for each reconstitution or recovery standard to confirm extraction efficiency, instrument performance, column integrity, chromatography, and mass calibration. Several of these reconstitution and recovery standards served as retention index (RI) markers and were checked for retention time and alignment. In-house software was used for peak detection and integration. The output from this processing generated a list of m/z ratios, retention times and area under the curve values. The software specified criteria for peak detection including thresholds for signal to noise ratio, height and width. Missing values, if any, were imputed with the observed minimum value for the given compound.

The sample sets, including QC samples, were chromatographically aligned based on a retention index that utilizes reconstitution standards assigned a fixed RI value. The RI of the experimental peak was determined by assuming a linear fit between flanking RI markers whose values do not change. The benefit of the RI is that it corrects for retention time drifts that are caused by systematic variations such as sample pH and column age. Each compound's RI was designated based on the elution relationship with its two lateral retention markers. Using an in-house software package, integrated, aligned peaks were matched against an in-house library (a chemical library) of authentic standards and routinely detected unknown compounds, which is specific to the four LC/MS methods described herein. Matches were based on retention index values, and the range of RI units varied relative to the LC/MS method. The experimental spectra were compared to the library spectra for the authentic standard and assigned forward and reverse scores. A perfect forward score would indicate that all ions in the experimental spectra were found in the library for the authentic standard at the correct ratios and a perfect reverse score would indicate that all authentic standard library ions were present in the experimental spectra and at correct ratios. The forward and reverse scores were compared, and a MS/MS fragmentation spectral score was given for the proposed match. All matches were then manually or automatically reviewed, and matches were approved or rejected. Each match was reviewed based on the mass, RI, and scores to assess the match, and the match was approved if the above criteria were met.

Further details regarding a chemical library, a method for matching integrated aligned peaks for identification of named compounds and routinely detected unknown compounds, and computer-readable code for identifying small molecules in a sample may be found in U.S. Pat. No. 7,561,975, which is incorporated by reference herein in its entirety.

### III. Quality Control.

Methods were put in place to control the quality of sample extraction and instrument run procedures. Technical replicates were made by combining aliquots of each of the individual test/experimental samples, or, for plasma samples, a pooled plasma sample from a commercial source was used. The technical replicate samples were extracted as described above. Extracts of the technical replicate samples were injected four times for each data set on each instrument to assess process variability. As an additional quality control, three water aliquots were also extracted as part of the sample set for each LC/MS method to serve as process blanks for artifact identification. All QC samples included the recovery and reconstitution standards for the given LC/MS method. The recovery and reconstitution standards were used to assess extraction efficiency and instrument performance and to serve as retention index markers for ion identification. The recovery and reconstitution standards were isotopically labeled or were non-labeled otherwise exogenous molecules chosen so as not to obstruct detection of intrinsic ions.

### Example 1: Liquid Chromatography/Mass Spectrometry (LC/MS) Method 1

Chromatography and mass spectrometry methods were developed to determine the presence, absence, or amount of one or more or a plurality of analytes in a single injection. A single fixed aliquot of 5.0 μL of the final extracted, reconstituted, sample was injected onto the UPLC column for each sample analyzed. A Waters Acquity UPLC system equipped with a fixed loop autosampler, two binary solvent managers for parallel column regeneration, and a column manager was used for liquid chromatography with a reversed phase column (Waters ACQUITY BEH C18, 2.1×100 mm 1.7 μm particle size). Mass spectrometry was performed on the sample extracts using a Thermo Q-Exactive mass spectrometer.

Liquid chromatography was performed on the samples. Mobile phase A was ammonium bicarbonate in water, and mobile phase B was ammonium bicarbonate in methanol and water. Linear gradient elution was carried out with an initial condition of 0.5% mobile phase B and 350 μL/min flow rate.

The eluent from the chromatography column, was directly and automatically introduced into the electrospray source of a mass spectrometer. The instrument was operated in negative ESI mode. Ionspray voltage was set at −3.2 kV, source temperature at 300° C., capillary temperature at 300° C., sheath gas at 70 units, auxiliary gas at 25 units, and S-lens RF at 40. The total run time was 6.5 minutes.

In one example, LC/MS Method 1 was developed to determine the presence, absence, or amount of one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 1-(14 or 15-methyl)palmitoyl-GPC (a17:0 or i17:0), 2-methoxyhydroquinone glucuronide (2), 2-methoxyhydroquinone sulfate (2), 3,5-dichloro-2,6-dihydroxybenzoic acid, 3-bromo-5-chloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-methylnonanoylcarnitine, 4-vinylguaiacol glucuronide, 5-hydroxy-2-methylpyridine sulfate, 5-hydroxyindole glucuronide, ascorbic acid 3-sulfate, azelaoyltaurine, butyrylputrescine, chenodeoxycholic acid sulfate (1), cortolone glucuronide, cyclo(ala-arg), cyclo(his-tyr), cyclo(his-val), dehydroandrosterone glucuronide, deoxycholic acid (12 or 24)-sulfate, deoxycholic acid glucuronide, dibutyl sulfosuccinate, glycoursodeoxycholic acid sulfate (1), hexanoyltaurine, isoursodeoxycholate sulfate (1), levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-methylbutanol glucuronide, 4-allylcatechol glucuronide, butyryltaurine, isobutyryltaurine, N-acetylserine-valine-arginine, and combinations thereof. The analytes were measured in control samples using LC/MS Method 1. The identity of all of the analytes measured using LC/MS Method 1 was confirmed by matching the Retention Index (RI), Mass, and MS/MS fragmentation pattern data of the analyte to the Retention Index (RI), Mass, and MS/MS fragmentation pattern data obtained with the corresponding authentic chemical standard.

### Example 2: Liquid Chromatography/Mass Spectrometry (LC/MS) Method 2

Chromatography and mass spectrometry methods were developed to determine the presence, absence, or amount of one or more or a plurality of analytes in a single injection. A single fixed aliquot of 5.0 μL of the final extracted sample was injected onto the UPLC column for each sample analyzed. A Waters Acquity UPLC system equipped with a fixed loop autosampler, two binary solvent managers for parallel column regeneration, and a column manager was used for liquid chromatography with a HILIC column (Waters ACQUITY BEH Amide, 2.1×150 mm 1.7 μm particle size). Mass spectrometry was performed on the sample extracts using a Thermo Q-Exactive mass spectrometer.

Liquid chromatography was performed on the samples. Mobile phase A was ammonium formate (pH 10.8), acetonitrile, methanol, and water, and mobile phase B was ammonium formate (pH 10.8) and acetonitrile. Linear gradient elution was carried out with an initial condition of 5% mobile phase B and a 500 μL/min flow rate.

The eluent from the chromatography column, was directly and automatically introduced into the electrospray source of a mass spectrometer. The instrument was operated in negative ESI mode. Ionspray voltage was set at −3.2 kV, source temperature at 300° C., capillary temperature at 300° C., sheath gas at 70 units, auxiliary gas at 20 units, and S-lens RF at 40. The total run time was 6.8 minutes.

In this example, LC/MS Method 2 was developed to determine the presence, absence, or amount of one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 2-methoxyhydroquinone sulfate (2), 3,5-dichloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-vinylguaiacol glucuronide, 5-hydroxy-2-methylpyridine sulfate, 5-hydroxyindole glucuronide, ascorbic acid 3-sulfate, azelaoyltaurine, dehydroandrosterone glucuronide, deoxycholic acid glucuronide, dibutyl sulfosuccinate, hexanoyltaurine, levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-hydroxyadipoylcarnitine, 3-methylbutanol glucuronide, 4-allylcatechol glucuronide, butyryltaurine, isobutyryltaurine, N-acetylserine-valine-arginine, and combinations thereof. The analytes were measured in control samples using LC/MS Method 2. The identity of all of the analytes measured using LC/MS Method 2 was confirmed by matching the Retention Index (RI), Mass, and MS/MS fragmentation pattern data of the analyte to the Retention Index (RI), Mass, and MS/MS fragmentation pattern data obtained with the corresponding authentic chemical standard.

### Example 3: Liquid Chromatography/Mass Spectrometry (LC/MS) Method 3

Chromatography and mass spectrometry methods were developed to determine the presence, absence, or amount of one or more or a plurality of analytes in a single injection. A single fixed aliquot of 5.0 μL of the final extracted sample was injected onto the UPLC column for each sample analyzed. A Waters Acquity UPLC system equipped with a fixed loop autosampler, two binary solvent managers for parallel column regeneration, and a column manager was used for liquid chromatography with a reversed phase column (Waters ACQUITY BEH C18, 2.1×100 mm 1.7 μm particle size). Mass spectrometry was performed on the sample extracts using a Thermo Q-Exactive mass spectrometer.

Liquid chromatography was performed on the samples. Mobile phase A was PFPA, formic acid, and water, and mobile phase B was PFPA, formic acid, and methanol. Linear gradient elution was carried out with an initial condition of 5% mobile phase B and a 350 μL/min flow rate.

The eluent from the chromatography column, was directly and automatically introduced into the electrospray source of a mass spectrometer. The instrument was operated in positive ESI mode. Ionspray voltage was set at 4.0 kV, source temperature at 300° C., capillary temperature at 250° C., sheath gas at 70 units, auxiliary gas at 15 units, and S-lens RF at 40. The total run time was 3.4 min.

In this example, LC/MS Method 3 was developed to determine the presence, absence, or amount of one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 2-iminopiperidine, 3-hydroxy-2-methylpyridine sulfate, 3-hydroxy-4-methylpyridine sulfate, 3-hydroxypyridine glucuronide, 4-ethylcatechol sulfate, 5-hydroxy-2-methylpyridine sulfate, ascorbic acid 3-sulfate, azelaoyltaurine, butyrylputrescine, dibutyl sulfosuccinate, hexanoyltaurine, levulinoylcarnitine, o-tyramine, phenylacetyl-beta-alanine, phenylacetyltaurine, phenylacetylvaline, 3-hydroxyadipoylcarnitine, butyryltaurine, isobutyryltaurine, N-acetylserine-valine-arginine, and combinations thereof. The analytes were measured in control samples using LC/MS Method 3. The identity of all of the analytes measured using LC/MS Method 3 was confirmed by matching the Retention Index (RI), Mass, and MS/MS fragmentation pattern data of the analyte to the Retention Index (RI), Mass, and MS/MS fragmentation pattern data obtained with the corresponding authentic chemical standard.

### Example 4: Liquid Chromatography/Mass Spectrometry (LC/MS) Method 4

Chromatography and mass spectrometry methods were developed to determine the presence, absence, or amount of one or more or a plurality of analytes in a single injection. A single fixed aliquot of 5.0 μL of the final extracted sample was injected onto the UPLC column for each sample analyzed. A Waters Acquity UPLC system equipped with a fixed loop autosampler, two binary solvent managers for parallel column regeneration, and a column manager was used for liquid chromatography with a reversed phase column (Waters ACQUITY BEH C18, 2.1×100 mm 1.7 μm particle size). Mass spectrometry was performed on the sample extracts using a Thermo Q-Exactive mass spectrometer.

Liquid chromatography was performed on the samples. Mobile phase A was PFPA, formic acid, and water, and mobile phase B was PFPA, formic acid, acetonitrile, and methanol. Linear gradient elution was carried out with an initial condition of 40% mobile phase B and a 600 μL/min flow rate.

The eluent from the chromatography column, was directly and automatically introduced into the electrospray source of a mass spectrometer. The instrument was operated in positive ESI mode. Ionspray voltage was set at 4.2 kV, source temperature at 400° C., capillary temperature at 350° C., sheath gas at 45 units, auxiliary gas at 30 units, and S-lens RF at 40. The total run time was 3.4 min.

In this example, LC/MS Method 4 was developed to determine the presence, absence, or amount of one or more or a plurality of analytes selected from the group consisting of N-butyryl-leucine, N-butyryl-phenylalanine, 1-(14 or 15-methyl)palmitoyl-GPC (a17:0 or i17:0), 3-hydroxymargaroylglycine, 4-methylnonanoylcarnitine, deoxycholic acid glucuronide, phenylacetylvaline, undecenoylcarnitine (C11:1), and combinations thereof. The analytes were measured in control samples using LC/MS Method 4. The identity of all of the analytes measured using LC/MS Method 4 was confirmed by matching the Retention Index (RI), Mass, and MS/MS fragmentation pattern data of the analyte to the Retention Index (RI), Mass, and MS/MS fragmentation pattern data obtained with the corresponding authentic chemical standard.

1-26. (canceled)

27. A method for determining in a sample, by liquid chromatography/mass spectrometry, the presence, absence, or amount of one or more or a plurality of analytes selected from the group consisting of N2-acetyl, N6,N6-dimethyllysine, N-butyryl-leucine, N-butyryl-phenylalanine, N-succinyl-leucine, N-succinyl-phenylalanine, o-tyramine, phenylacetyl-beta-alanine, phenylacetyltaurine, phenylacetylvaline, 5-hydroxyindole glucuronide, chenodeoxycholic acid sulfate (1), deoxycholic acid (12 or 24)-sulfate, deoxycholic acid glucuronide, glycoursodeoxycholic acid sulfate (1), isoursodeoxycholate sulfate (1), ascorbic acid 3-sulfate, 1-(14 or 15-methyl)palmitoyl-GPC (a17:0 or i17:0), 3-hydroxyadipoylcarnitine, 3-hydroxymargaroylglycine, 4-methylnonanoylcarnitine, azelaoyltaurine, butyryltaurine, hexanoyltaurine, isobutyryltaurine, levulinoylcarnitine, undecenoylcarnitine (C11:1), 3,5-dichloro-2,6-dihydroxybenzoic acid, 3-bromo-5-chloro-2,6-dihydroxybenzoic acid, 2-methoxyhydroquinone glucuronide (2), 2-methoxyhydroquinone sulfate (2), 4-allylcatechol glucuronide, 4-allylcatechol sulfate, 4-ethylcatechol sulfate, 3-hydroxy-2-methylpyridine sulfate, 3-hydroxy-4-methylpyridine sulfate, 3-hydroxypyridine glucuronide, 5-hydroxy-2-methylpyridine sulfate, 2-iminopiperidine, thymidine sulfate (2), cyclo(ala-arg), cyclo(his-tyr), cyclo(his-val), N-acetylserine-valine-arginine, 4-vinylguaiacol glucuronide, maltol sulfate, methyl vanillate sulfate, butyrylputrescine, 5-androsten-3b,16a,17b-triol sulfate (1), 5-androsten-3b,16b,17a-triol sulfate (1), 5-androstenetriol disulfate, cortolone glucuronide, dehydroandrosterone glucuronide, (2-butoxyethoxy)acetic acid, dibutyl sulfosuccinate, 3-methylbutanol glucuronide, and combinations thereof, the method comprising:
a) subjecting the sample to an ionization source under conditions suitable to produce one or more ions detectable by mass spectrometry from each of the one or more of the analytes;
b) measuring, by mass spectrometry, the amount of the one or more ions from each of the one or more analytes; and
c) using the measured amount of the one or more ions to determine the presence, absence, or amount of each of the one or more or plurality of analytes in the sample.

28. The method of claim 27, wherein the one or more or plurality of analytes are selected from the group consisting N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 1-(14 or 15-methyl)palmitoyl-GPC (a17:0 or i17:0), 2-methoxyhydroquinone glucuronide (2), 2-methoxyhydroquinone sulfate (2), 3,5-dichloro-2,6-dihydroxybenzoic acid, 3-bromo-5-chloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-methylnonanoylcarnitine, 4-vinylguaiacol glucuronide, 5-hydroxy-2-methylpyridine sulfate, 5-hydroxyindole glucuronide, ascorbic acid 3-sulfate, azelaoyltaurine, butyrylputrescine, chenodeoxycholic acid sulfate (1), cortolone glucuronide, cyclo(ala-arg), cyclo(his-tyr), cyclo(his-val), dehydroandrosterone glucuronide, deoxycholic acid (12 or 24)-sulfate, deoxycholic acid glucuronide, dibutyl sulfosuccinate, glycoursodeoxycholic acid sulfate (1), hexanoyltaurine, isoursodeoxycholate sulfate (1), levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-methylbutanol glucuronide, 4-allylcatechol glucuronide, butyryltaurine, isobutyryltaurine, N-acetylserine-valine-arginine, and wherein the presence, absence, or amount of the one or more or plurality of analytes are determined in a single injection.

29. The method of claim 27, wherein the one or more or plurality of analytes are selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 2-methoxyhydroquinone sulfate (2), 3,5-dichloro-2,6-dihydroxybenzoic acid, 3-hydroxy-2-methylpyridine sulfate, 3-hydroxy-4-methylpyridine sulfate, 3-hydroxymargaroylglycine, 3-hydroxypyridine glucuronide, 4-allylcatechol sulfate, 4-ethylcatechol sulfate, 4-vinylguaiacol glucuronide, 5-hydroxy-2-methylpyridine sulfate, 5-hydroxyindole glucuronide, ascorbic acid 3-sulfate, azelaoyltaurine, dehydroandrosterone glucuronide, deoxycholic acid glucuronide, dibutyl sulfosuccinate, hexanoyltaurine, levulinoylcarnitine, maltol sulfate, methyl vanillate sulfate, o-tyramine, phenylacetyl-beta-alanine, phenylacetyltaurine, phenylacetylvaline, thymidine sulfate (2), 5-androsten-3b,16a,17b-triol sulfate (1), 5-androstenetriol disulfate, 5-androsten-3b,16b,17a-triol sulfate (1), 3-hydroxyadipoylcarnitine, 3-methylbutanol glucuronide, 4-allylcatechol glucuronide, butyryltaurine, isobutyryltaurine, N-acetylserine-valine-arginine, and wherein the presence, absence, or amount of the one or more or plurality of analytes are determined in a single injection.

30. The method of claim 27, wherein the one or more or plurality of analytes are selected from the group consisting of N2-acetyl, N6,N6-dimethyllysine, N-butyryl-leucine, N-butyryl-phenylalanine, N-succinyl-leucine, N-succinyl-phenylalanine, (2-butoxyethoxy)acetic acid, 2-iminopiperidine, 3-hydroxy-2-methylpyridine sulfate, 3-hydroxy-4-methylpyridine sulfate, 3-hydroxypyridine glucuronide, 4-ethylcatechol sulfate, 5-hydroxy-2-methylpyridine sulfate, ascorbic acid 3-sulfate, azelaoyltaurine, butyrylputrescine, dibutyl sulfosuccinate, hexanoyltaurine, levulinoylcarnitine, o-tyramine, phenylacetyl-beta-alanine, phenylacetyltaurine, phenylacetylvaline, 3-hydroxyadipoylcarnitine, butyryltaurine, isobutyryltaurine, N-acetylserine-valine-arginine, and wherein the presence, absence, or amount of the one or more or plurality of analytes are determined in a single injection.

31. The method of claim 27, wherein the one or more or plurality of analytes are selected from the group consisting of N-butyryl-leucine, N-butyryl-phenylalanine, 1-(14 or 15-methyl)palmitoyl-GPC (a17:0 or i17:0), 3-hydroxymargaroylglycine, 4-methylnonanoylcarnitine, deoxycholic acid glucuronide, phenylacetylvaline, undecenoylcarnitine (C11:1), and wherein the presence, absence, or amount of the one or more or plurality of analytes are determined in a single injection.

32. The method of claim 27, wherein the one or more or plurality of analytes are selected from the group consisting of N2-acetyl, N6,N6-dimethyllysine, N-butyryl-leucine, N-butyryl-phenylalanine, N-succinyl-leucine, N-succinyl-phenylalanine, o-tyramine, phenylacetyl-beta-alanine, phenylacetyltaurine, phenylacetylvaline, 5-hydroxyindole glucuronide, and combinations thereof.

33. The method of claim 27, wherein the one or more or plurality of analytes are selected from the group consisting of chenodeoxycholic acid sulfate (1), deoxycholic acid (12 or 24)-sulfate, deoxycholic acid glucuronide, glycoursodeoxycholic acid sulfate (1), isoursodeoxycholate sulfate, and combinations thereof.

34. The method of claim 27, wherein the one or more or plurality of analytes are selected from the group consisting of 3-hydroxyadipoylcarnitine, 3-hydroxymargaroylglycine, 4-methylnonanoylcarnitine, azelaoyltaurine, butyryltaurine, hexanoyltaurine, isobutyryltaurine, levulinoylcarnitine, undecenoylcarnitine (C11:1), and combinations thereof.

35. The method of claim 27, wherein the one or more or plurality of analytes are selected from the group consisting of 3,5-dichloro-2,6-dihydroxybenzoic acid, 3-bromo-5-chloro-2,6-dihydroxybenzoic acid, 2-methoxyhydroquinone glucuronide (2), 2-methoxyhydroquinone sulfate (2), 4-allylcatechol glucuronide, 4-allylcatechol sulfate, 4-ethylcatechol sulfate, 3-hydroxy-2-methylpyridine sulfate, 3-hydroxy-4-methylpyridine sulfate, 3-hydroxypyridine glucuronide, 5-hydroxy-2-methylpyridine sulfate, and combinations thereof.

36. The method of claim 27, wherein the one or more or plurality of analytes are selected from the group consisting of N-acetylserine-valine-arginine, cyclo(ala-arg), cyclo(his-tyr), cyclo(his-val), and combinations thereof.

37. The method of claim 27, wherein the one or more or plurality of analytes are selected from the group consisting of 4-vinylguaiacol glucuronide, maltol sulfate, methyl vanillate sulfate, and combinations thereof.

38. The method of claim 27, wherein the one or more or plurality of analytes are selected from the group consisting of 5-androsten-3b,16a,17b-triol sulfate (1), 5-androsten-3b,16b,17a-triol sulfate (1), 5-androstenetriol disulfate, cortolone glucuronide, dehydroandrosterone glucuronide, and combinations thereof.

39. The method of claim 27, wherein the one or more or plurality of analytes are selected from the group consisting of 3-methylbutanol glucuronide, 2-iminopiperidine, (2-butoxyethoxy)acetic acid, dibutyl sulfosuccinate, and combinations thereof.

40. The method of claim 28, wherein the ionization source is operated in negative ionization mode.

41. The method of claim 30, wherein the ionization source is operated in positive ionization mode.

42. The method of claim 27, wherein the amount of two or more analytes is determined.

43. The method of claim 27, wherein the amount of ten or more analytes is determined.

44. The method of claim 27, wherein the amount of 25 or more analytes is determined.

45. The method of claim 27, wherein the amount of 55 or more analytes is determined.

46. The method of claim 27, wherein said liquid chromatography is selected from the group consisting of high performance liquid chromatography, ultra high performance liquid chromatography, and turbulent flow liquid chromatography.
